(19) United States
(12) Patent Application Publication
(10) Pub. No.: US 2018/0250232 A1
(43) Pub. Date: Sep. 6, 2018

(54) PROCESS FOR PREPARING SPRAY DRIED SOLID DISPERSIONS OF (S)-N-(3-(6-ISOPROPOXYPYRIDIN-3-YL)-1H-INDAZOL-5-YL)-1-(2-(4-(4-(1-METHYL-1H-1,2,4-TRIAZOL-3-YL)PHENYL)-3,6-DIHYDROPYRIDIN-1(2H)-YL)-2-OXOETHYL)-3-(METHYLTHIO)PYRROLIDINE-3-CARBOXAMIDE FOR PHARMACEUTICAL PREPARATIONS

(71) Applicants: Yung-Chi LEE, Kenilworth, NJ (US); Dan ZHANG, Rahway, NJ (US); Merck Sharp & Dohme Corp., Rahway, NJ (US)

(72) Inventors: Yung-Chi Lee, New Providence, NJ (US); Dan Zhang, Watchung, NJ (US)

(73) Assignee: Merck Sharp & Dohme Corp., Rahway, NJ (US)

(21) Appl. No.: 15/756,630

(22) PCT Filed: Aug. 29, 2016
(86) PCT No.: PCT/US16/49175

§ 371 Date: Mar. 1, 2018

Related U.S. Application Data
(60) Provisional application No. 62/213,671, filed on Sep. 3, 2015.
(60) Provisional application No. 62/302,906, filed on Mar. 3, 2016.

(51) Int. Cl. A61K 9/16; A61K 31/444

(52) U.S. Cl. CPC A61K 9/1652; A61K 31/444; A61K 9/1694

(57) ABSTRACT

A process for preparing spray dried solid dispersions of (S)—N-(3-(6-isopropoxypyridin-3-yl)-1H4ndazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base and hypromellose acetate succinate for pharmaceutical preparations including pharmaceutical capsule preparations.

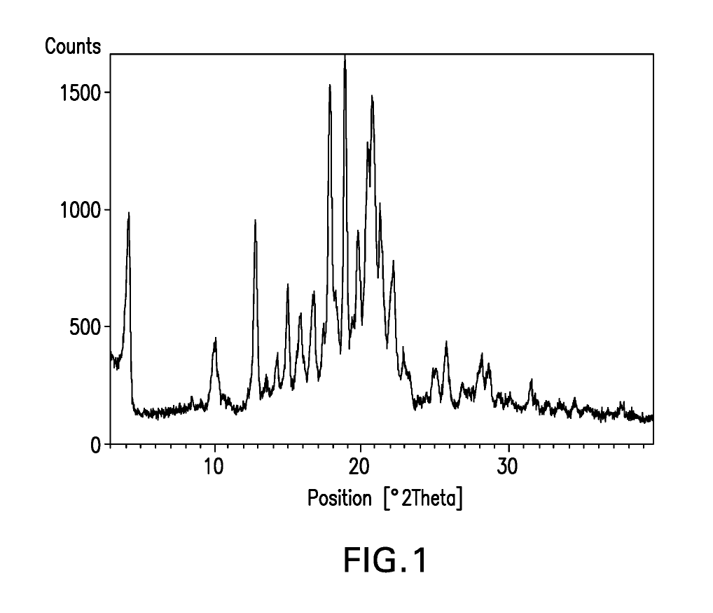

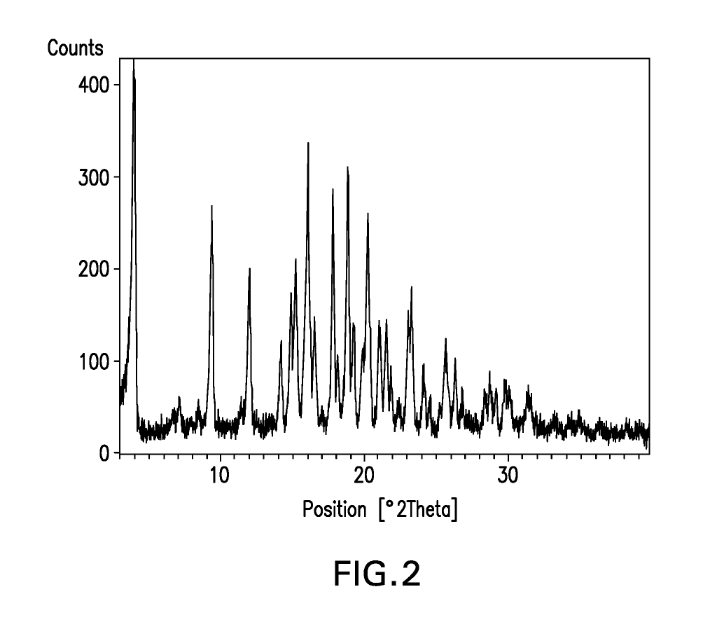

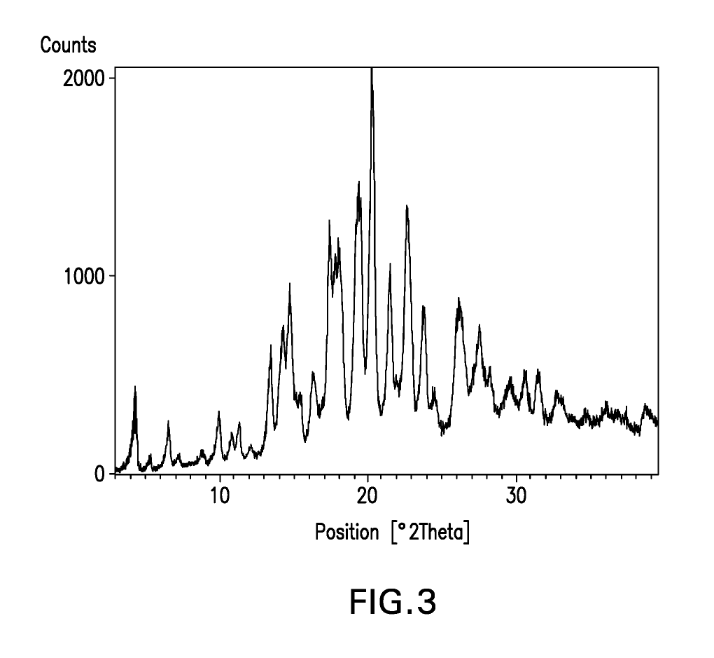

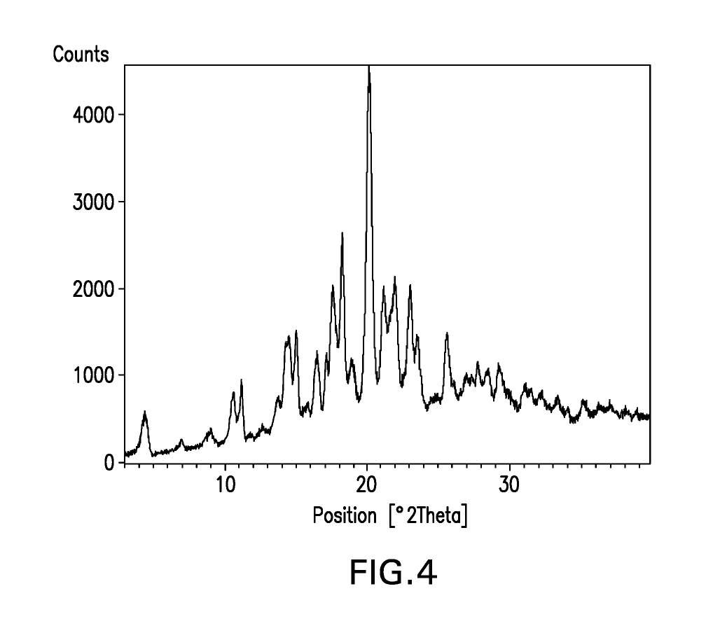

### BACKGROUND OF THE INVENTION

WO2009/105500 describes ERK inhibitors, including procedures for making them, and procedures for preparing pharmaceutical compositions comprising them. Described pharmaceutical compositions include solid form preparations including powders, tablets, dispersible granules, capsules, cachets and suppositories for direct administration to a patient; liquid form preparations including solutions, suspensions and emulsions for direct administration to a patient; aerosol preparations suitable for inhalation; solid form preparations which are intended to be converted, shortly before use, to liquid form preparations, including solutions, suspensions and emulsions for subsequent administration to a patient; and transdermal compositions including creams, lotions, aerosols and/or emulsions for direct application to the patient or administration via transdermal patch.

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide, a specific ERK inhibitor described in WO2009/105500, can exist in several crystalline forms as well as amorphous form. During formulation processing, certain forms, such as (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline form 1 HCl, can become converted into other forms. For example, depending on process conditions, roller compaction of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline form 1 HCl can result in creation of significant amounts of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide amorphous free base, and mechanical stress can result in creation of significant amounts of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide amorphous HCl. In order to efficiently prepare safe and effective oral (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline form 1 HCl pharmaceutical tablet and capsules compositions for administration to patients, it is highly desirable to create (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide granules that minimize conversion from the crystalline form 1 HCl to the amorphous free base and amorphous HCl salt form. The known forms have different aqueous solubilities and dissolution profiles and could pose a high risk to in-vivo performance if not controlled.

The present invention provides a spray dried solid dispersion of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide amorphous free base which are used for efficient preparation of tablets and capsules comprising (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide that is suitable for safe and effective oral administration to a patient.

### SUMMARY OF THE INVENTION

The invention is a process for preparing a solid spray-dried dispersion comprising (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide and hypromellose acetate succinate, comprising the steps of
a) blending (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline free base hydrate form 2 and hypromellose acetate succinate to form a slurry, and
b) spraying the slurry in a spray drier under conditions sufficient to dry the slurry and form a solid dispersion of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base and hypromellose acetate succinate.

In one embodiment, the weight ratio of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline free base hydrate form 2 and hypromellose acetate succinate is between about 1:1 and 1:5.

In one embodiment, the weight ratio is about 1:3.

The invention is also a solid spray-dried dispersion comprising (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide free base hydrate form 2 and hypromellose acetate succinate in a weight ratio of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide crystalline free base hydrate form 2 to hypromellose acetate succinate of about 1:3.

### DETAILED DESCRIPTION OF THE INVENTION

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide, structure I below:

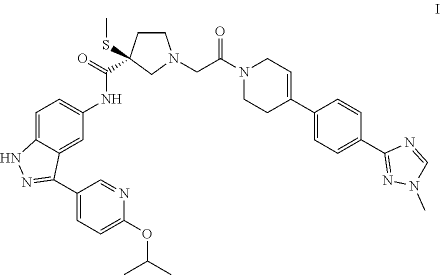 [SMILES: CS[C@@]1(C(=O)Nc2ccc3[nH]nc(-c4ccc(OC(C)C)nc4)c3c2)CCN(CC(=O)N2CC=C(c3ccc(-c4ncn(C)n4)cc3)CC2)C1]

and method for its preparation, is described in patent publication WO2009/105500 (compound A6). (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide is also available from Active Biochem CAT# A-1191. The compound, which inhibits ERK activity (i.e., ERK1 and ERK2 activity), may be useful for treating a broad spectrum of cancers, such as, for example, melanoma, pancreatic cancer, thyroid cancer, colorectal cancer, lung cancer, breast cancer, and ovarian cancer.

### Preparation

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide synthesis is 19 steps. Compound preparation is divided into three intermediate preparations A, B and C followed by coupling of the intermediates. All intermediates start with commercially available compounds. Compound 5 is prepared by reaction of the commercially available bromo-4-cyanobenzene with methyl hydrazine under acidic conditions to form the hydrazinoimidate 2 in modest yield. After reaction with formic acid in two steps the bromophenyl-N-methyl triazole intermediate 3 is obtained. The tetrahydropyridine ring is introduced by a Suzuki reaction of the commercially available Boc protected tetrahydropyridine-boronate to obtain the tricyclic ring system 4. Chloroacetamide 5 is obtained in excellent yield by reaction of the deprotected 4 with chloroacetylchloride. The pyrrolidine core 10a is obtained in good yield in 5 steps starting from commercially available 6. Reaction with thionylchloride gave the thiomethyl olefin 7. Cycloaddition (2+3) gave 8 followed by removal of the benzyl protection group to give 9. L-Tartaric acid resolution of the pyrrolidine core gives the pure (S) enantiomer 9 after filtration from methanol. After protection as the Boc derivative and hydrolysis of the methyl ester, 10 is obtain in overall 50% yield. Compound 17 is obtained from commercially available indazole 11. Bromination at the 3-position of indazole 11 proceeded in excellent yield without chromatography to obtain 12. Suzuki reaction of the bromo compound 12 with 14 gave the nitro indazole 16 after chromatography. Reduction of 16 gave aniline 17 as an oil in quantitative yield without chromatography. The final coupling of the intermediates proceeded by coupling 17 with 10a to obtain 18 in good yield. After deprotection of the Boc and Trityl groups the final coupling with 5 gave (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide after chromatography. Final purification was carried out by crystallization from methanol/diethylether. This synthetic route has been conducted on a scale that delivered (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide.

### Synthesis From Key Intermediates 5, 10 and 17

### Preparation A

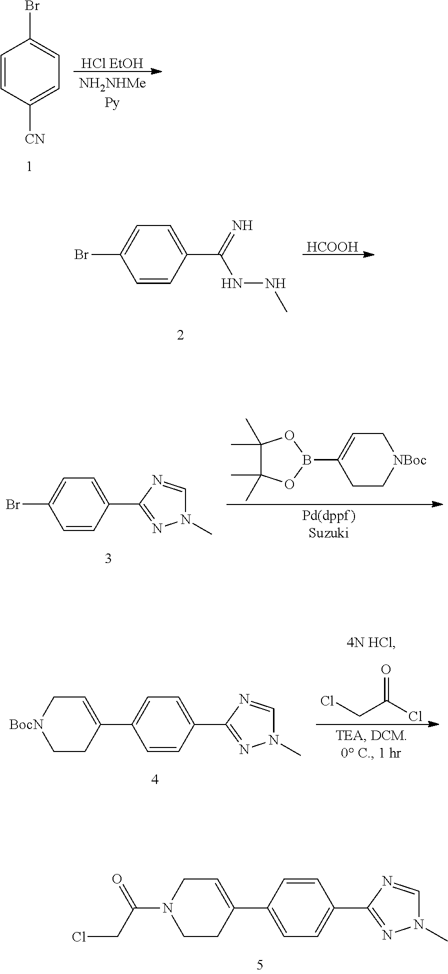

### Preparation B

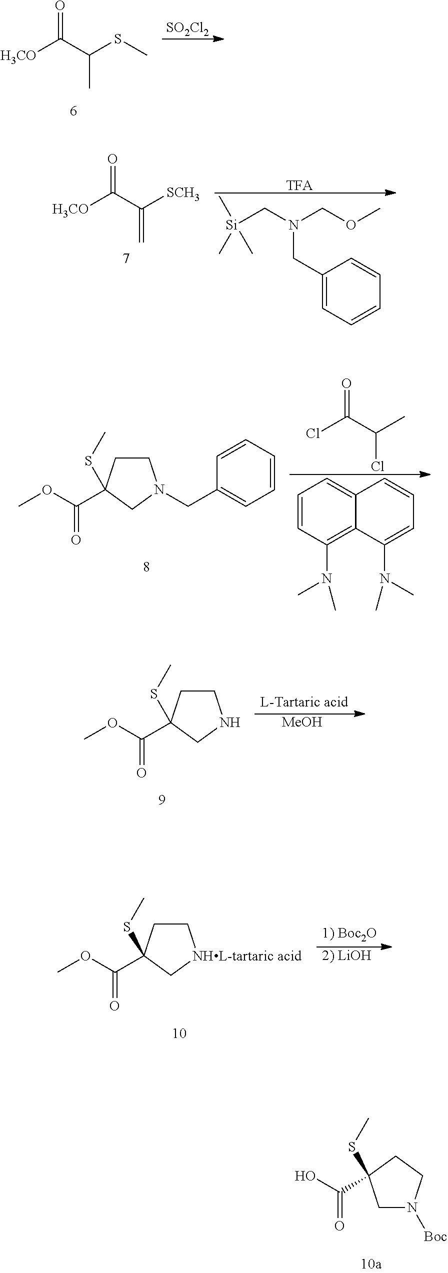

### Preparation C

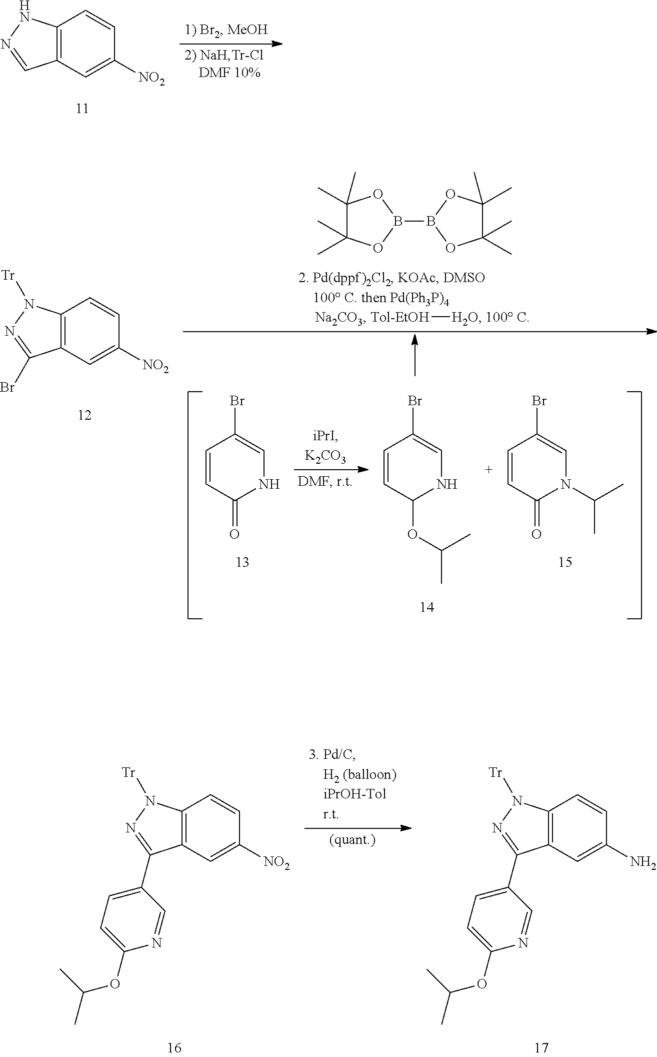

### Final Coupling:

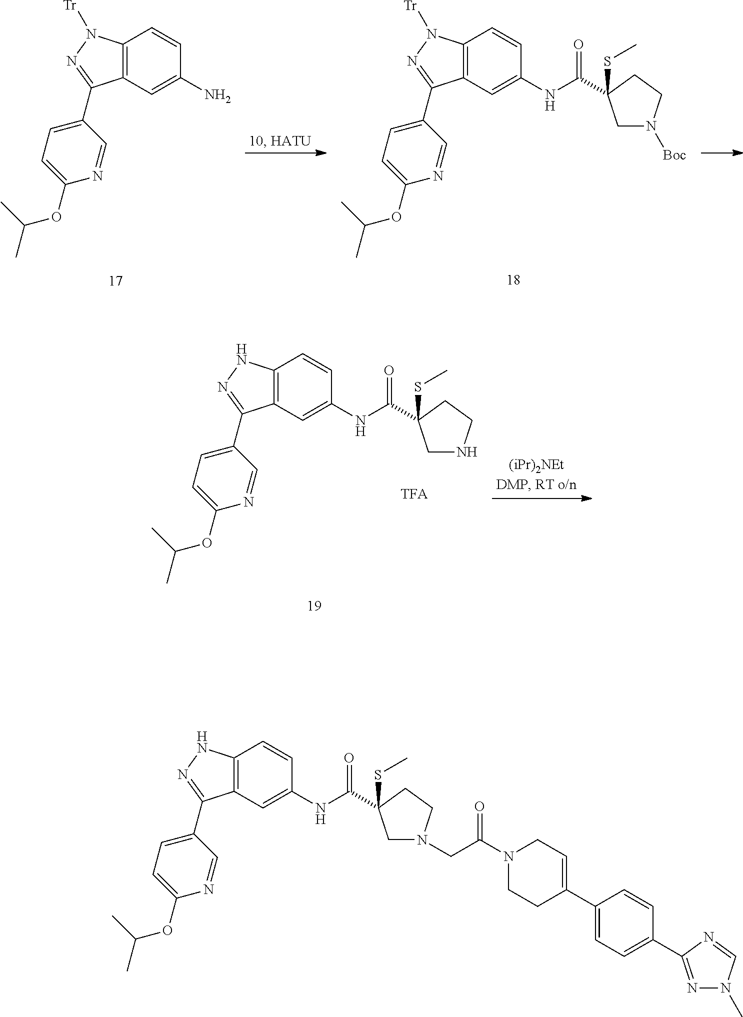

### Free Base Hydrate Form 1 and Free Base Hydrate Form 2

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline free base hydrate form 2 is the most stable crystalline form at room temperature based on results of polymorph studies. Preliminary polymorph screening for (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide were conducted by solvent-mediated slurry experiments in 10 different solvents or solvent mixtures using the amorphous compound as the starting material.

Two crystalline free base forms were observed in the study, they were hydrate form 1 and hydrate form 2. Hydrate form 1 was identified in slurry samples of methanol, and H₂O/methanol mixture (with H₂O activity of 0.5), while hydrate form 2 was identified in slurry sample of acetonitrile. The melting point of hydrate form 2 is 133° C.

To compare the two forms, competition slurry experiments using them as seeds in H₂O/methanol mixture (with H₂O activity of 0.5) were conducted at room temperature and 4° C., the results showed that hydrate form 2 was the more stable form in both experiments.

Polymorph studies showed a complex picture for (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide. Form screening experiments also enabled observation of several other crystalline forms, including ethanol solvate, acetone solvate, and two other forms crystallized in methanol. Further studies are continuing to characterize the forms and to understand the inter-conversion mechanism. Hydrate form 2 was characterized to be a mono-hydrate, based on the results of TGA and moisture analysis (by Karl-Fisher Moisture Analyzer). Hydrate form 2 is an acceptable form for FIH development based on current polymorph, stability, and in vivo animal PK results. In rats, hydrate form 2 showed comparable PK exposure levels to those of the amorphous compound.

### Spray Drying

Spray drying involves transformation of a formulation from a fluid state into a dried form by spraying the formulation into a hot drying medium. The formulation can be either a solution, suspension, or a paste. The spray dried product is typically in the form of a powder consisting of single particles or agglomerates, depending upon the physical and chemical properties of the formulation and the dryer design and operation. The basic technique includes the following four steps: a) atomization of the formulation solution into a spray; b) spray-gas contact; c) drying of the spray; and d) separation of the dried product from the drying gas.

The invention provides methods and formulations for providing a spray dried product that can be used to fill capsule preparations. The pharmaceutical solution to be spray dried is preferably selected so as to provide (upon spray drying) a substantially uniform powder with a favorable moisture content and reconstitution profile.

The process for spray drying a pharmaceutical formulation of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide is preferably undertaken aseptically. Prior to the spray drying step, a solution compounding system may be used to rapidly mix the active pharmaceutical ingredient (API) and excipients in an environmentally controlled system, including one or more polymers selected from HPMCAS-MF (hypromellose acetate succinate, MF grade), HPMCAS-HF (hypromellose acetate succinate, HF grade) and VA64 (polyvinylpyrolidone/vinyl acetate copolymer). Hypromellose and hydroxypropyl methylcellulose are synonymous.

In a preferred embodiment of the process, the polymers are selected from HPMCAS-MF (hypromellose acetate succinate, MF grade) and HPMCAS-HF.

The system can be a conventional tank and mixer blending system and should minimize operator exposure to the API, and minimize the API's exposure to air/oxygen during the handling, mixing and holding of ingredients prior to spray drying in order to avoid premature degradation of the API if susceptible to oxidation. The compounded solution undergoes a sterile filtration process prior to the aseptic spray drying process. Following the spray drying process, the resulting powder is harvested into sterile vessels. At the appropriate time, dried powder is then transferred to a filler that fills the resulting spray dried (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide in pharmaceutical vials, which are then capped and sealed.

The process may utilize pre-cooled, nitrogen-purged WFI that is added to a nitrogen purged tank through a flow meter to measure the quantity of water. Since the process may be weight based, the full quantity of water, which will be based on the weight of the API to be mixed, can be charged initially.

Once compounding activities have been completed and the mixed solution has met the pre-defined chemistry limits (e.g., pH), the solution is transferred to the spray dryer. The actual spray drying involves the atomization of a liquid solution (feedstock) into a spray of droplets, and contacting the droplets with hot gas in a drying chamber. The droplets can be produced by, for example, nozzle atomizers. Evaporation of moisture from the produced droplets and formation of dry particles proceed under controlled temperature and gas flow conditions. When the droplets are small enough and the chamber large enough, the droplets dry before they reach the wall of the chamber. The resulting product is collected as a free-flowing material. Powder is discharged continuously from the drying chamber. Operating conditions and spray dryer design are selected according to the drying characteristics of the product and powder specification.

Spray dryers generally include a feedstock pump, an atomizer, a gas heater, a gas disperser, a drying chamber, and systems for exhaust gas cleaning and powder recovery. An example spray drying system includes drying gas introduced into a pre-filter. In one aspect, the drying gas is nitrogen, and avoids the presence of oxygen. In one embodiment, the drying gas comprises nitrogen with less than 1% oxygen. The drying gas then passes through a fan and a heater, which may be an electric heater. The drying gas then passes through a sterilizing gas filter and an inlet gas temperature gauge monitors the inlet gas temperature before it is introduced into a drying chamber via a ceiling gas dispenser. Redundant filtration may be employed to ensure product quality.

The formulation, from the tank and mixer blending system, undergoes a sterile filtration process prior to being fed into the drying chamber and atomized by an atomizer. The atomizer may be any type of known atomizer that allows for aseptic processing such as a pressure nozzle or a two-fluid nozzle (e.g., available from GEA Process Engineering Inc., Columbia, Md., formerly known as Niro Inc.). The atomizer disperses the liquid formulation into a controlled drop size spray. In one particular embodiment, the atomizer is operated with a nozzle protection of 80 kg/hour nitrogen gas at 80° C. The spray is then heated in the drying chamber. The heated drying gas evaporates the liquid from the spray and forms dry particles.

After the solution has been atomized and heated, the dry particles exit the drying chamber and proceed into a cyclone, which separates the powder from the gas. The powder flows out of the cyclone at outlet into a sterile powder collection vessel and the rest of the gas flows out past an outlet gas temperature gauge toward a cartridge filtration system. The cartridge filtration system removes fine particles at outlet. The remaining dried gas then flows through a second filter (e.g. a sterile filter), and in some embodiments through a third filter, and then back into the drying gas supply at. In one embodiment, a vortex eliminator may be used near the bottom of the cyclone to eliminate hot gas from passing through the outlet.

In various embodiments, the spray dryer may utilize a drying gas having an inlet temperature of about 150° C.-250° C., and preferably of about 170° C.-230° C. The drying gas may have an outlet temperature of about 70° C.-150° C., and preferably of about 70° C.-120′C, for example the outlet temperature during the production of Azithromycin is 75. The nitrogen gas flow rate can range from 650-750 kg/hour but other flow rates can be used to accommodate the rate of the feedstock, equipment and temperature variations.

### Example 1

### Spray Dried Solid Dispersions of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide for Pharmaceutical Capsule Preparations

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline free base hydrate form 2 was incorporated into a water soluble polymer (one of hypromellose acetate succinate, MF grade or hypromellose acetate succinate, HF grade) by a solid dispersion approach utilizing a spray-drying process to prepare (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base solid dispersion.

Polymer plus (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-(2H)-yl)-2-oxoethyl)-3-(methylthio)pyrrolidine-3-carboxamide crystalline free base hydrate form 2 were added to acetone. The mixture was stirred for 40 minutes at room temperature at a rotational speed of 400 rpm on a magnetic stir plate or submersed into a Branson Ultra-Sonicator until all components had dissolved. A ProCepT Micro-Spray Dryer with a bifluid nozzle, with an aperture of 0.6 mm, was used this study. Spraying conditions included inlet temperature of 85±3° C. and outlet temperature of 53±5° C. Drying gas (air) flow rate was 0.35±0.01 m³/min.

### Capsule Preparation

Capsules comprising the following ingredients were prepared using conventional capsule preparation technology:

TABLE 1

| Material Description | Amounts | |
|---|---|---|
| (S)-N-(3-(6-isopropoxypyridin-3-yl)-1H-indazol-5-yl)-1- | 159.6 | mg |
| (2-(4-(4-(1-methyl-1H-1,2,4-triazol-3-yl)phenyl)-3,6- | | |
| dihydropyridin-1(2H)-yl)-2-oxoethyl)-3- | | |
| (methylthio)pyrrolidine-3-carboxamide | | |
| crystalline HCl salt form 1 | | |
| Lactose, Monohydrate, Compendial | 122.25 | mg |
| Microcrystalline Cellulose, Compendial | 122.25 | mg |
| Crospovidone, Compendial | 22.2 | mg |
| Silicon Dioxide, Colloidal, Compendial | 8.85 | mg |
| Stearic Acid, Compendial | 6.6 | mg |
| Stearic Acid, Compendial | 2.25 | mg |
| Fill weight | 444 | mg |

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline HCl salt form 1 can be replaced in the formula above in the same amounts, substantially the same amounts, or pharmaceutically acceptable different amounts, with (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base to form capsules containing dispersions of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide amorphous free base.

### Test Results

Table 2 shows that the solid dispersions were superior to the crystalline HCl salt in both in-vitro and in-vivo performance. The solid dispersion resulted in improved dissolution, as shown by concentration (ug/mL) range of various preparations over a period of dissolution time between 70 minutes and 160 minutes. In Table 2, “SD” refers to spray dried dispersion of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base. “Crystalline HCl salt Form 1” refers to (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide crystalline HCl salt form 1.

TABLE 2

| | Concentration |
|---|---|
| Preparation | (ug/mL) range |
| SD25% HPMCAS-MF | 160-180 |
| SD25% HPMCAS-HF | 100-110 |
| SD40% HPMCAS-HF | 55-65 |
| SD40% VA64 | 20-30 |
| Crystalline HCl salt form 1 | 20-30 |

(S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base precipitated from all preparations once the pH of the dissolution media changed from acidic to a neutral environment. The amount of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide amorphous free base remaining in solution can be ranked as

[25% drug load in HPMCAS-MF]>[25% drug load in HPMCAS-HF]>[40% drug load in HPMCAS-HIF]>[40% drug load in VA64]=[HCl salt]

(VA is polyvinylpyrolidone/vinyl acetate copolymer). This data set indicates that the use of pH dependent polymers can reduce the amount of drug precipitated. Additionally, it shows that the 25% drug load in HPMCAS-MF outperformed other solid dispersion preparations.

Solid dispersion also provided improved exposure in a pre-clinical animal model. Dog PK data of a 25% drug load in HPMCAS-MF (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base solid dispersion compared to (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline HCl salt form 1 in a capsule is tabulated in Table 3. “SDD” refers to spray dried dispersion of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base. “Dry filled capsule HCl form 1” refers to dry filled capsules containing (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline HCl salt form 1.

TABLE 3

| | Dose | AUC_{0-24 hr} | Cₘₐₓ | Tₘₐₓ | AUC |
|---|---|---|---|---|---|
| Formulation | (mpk) | (μM × hr) | (μM) | (hr) | ratio |
| 25% drug load in | 15 | 14.7 ± | 2.62 ± | 2.0 | 1.58 |
| HPMCAS-MF | | 2.13 | 0.19 | (2.0-4.0)* | |
| SDD | | | | | |
| Suspension (n = 6) | | | | | |
| Dry filled capsule | 15 | 9.35 ± | 1.82 ± | 2.0 | 1.00 |
| HCl form 1 | | 1.34 | 0.34 | (1.0-2.0)* | |
| (Benchmark, n = 6) | | | | | |

*For Tₘₐₓ, median value is provided along with the range in parenthesis.

The spray-dried solid dispersion utilizing HPMCAS-MF proved advantageous by reducing the precipitation tendency of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide, thereby improving in-vivo exposure.

What is claimed is:

1. A process for preparing a solid spray-dried dispersion comprising (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide and hypromellose acetate succinate, comprising the steps of
a) blending (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-(2H)-yl)-2-oxoethyl)-3-(methylthio)pyrrolidine-3-carboxamide crystalline free base hydrate form 2 and hypromellose acetate succinate to form a slurry, and
b) spraying the slurry in a spray drier under conditions sufficient to dry the slurry and form a solid dispersion of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide amorphous free base and hypromellose acetate succinate.

2. A process of claim 1 wherein the weight ratio of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide crystalline free base hydrate form 2 to hypromellose acetate succinate is between about 1:1 and 1:5.

3. A process of claim 2, wherein the weight ratio is about 1:3.

4. A solid spray-dried dispersion comprising (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1 (2H)-yl)-2-oxoethyl)-3-(methylthio)pyrrolidine-3-carboxamide free base hydrate form 2 and hypromellose acetate succinate in a weight ratio of (S)—N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide crystalline free base hydrate form 2 to hypromellose acetate succinate of about 1:3.
